(19) United States
(12) Patent Application Publication
(10) Pub. No.: US 2016/0296644 A1
(43) Pub. Date: Oct. 13, 2016

(54) ANTICANCER DRUG, COMPRISING A COPPER RADIOISOTOPE

(71) Applicant: Gianluca VALENTINI, Macerata (IT)

(72) Inventor: Gianluca VALENTINI, Macerata (IT)

(21) Appl. No.: 15/038,258

(22) PCT Filed: Oct. 24, 2014
(86) PCT No.: PCT/IB2014/002261

§ 371 Date: May 20, 2016

(30) Foreign Application Priority Data
Nov. 21, 2013 (IT) AN2013A000219

(51) Int. Cl. A61K 51/02

(52) U.S. Cl. CPC A61K 51/02

(57) ABSTRACT

The object of the present invention is a drug for the therapeutic treatment of tumours, also usable as diagnostic agent and therapeutic agent for the Alzheimer's disease.

Such drug comprises a copper-64 radioisotope in its salt form, said drug consisting of copper-64 chloride (64CuCl2).

The object of the present invention is a drug for the direct treatment of tumours and for the diagnosis and treatment of the Alzheimer's disease.

The invention falls within the field of anti-cancer drugs using the radioactive properties of certain chemical elements, capable of acting as radionuclides in the form of radiotracers or radiopharmaceuticals, in order to diagnose various pathologies or destroy cancer cells.

In particular, the subject drug uses the radionuclidic properties of copper-64 isotope, for a direct use as copper salt in the treatment of cancer forms and in the diagnosis and treatment of the Alzheimer's disease.

In the field of nuclear medicine, the last 20 years have seen a strong development in the use of the radionuclides, starting from technetium-99m isotope, which represents the most used tracer in SPECT (Single Photon Emission Computed Tomography) diagnostics.

Other radionuclides widely used are iodine-131, especially in the thyroid tumour therapy, and the fluorodeoxyglucose (FDG) in PET (Positron Emission Tomography) diagnostics, which currently represents the most used methodology within the medical-nuclear sphere for the staging and control of oncological therapeutic effects.

In the last two decades the role of copper in the body metabolism and its close relation with the progress of tumoural growth has been widely described.

In fact, copper has a substantial role in the angiogenesis mechanism and in the DNA replication, besides the progress mechanisms of other physiopathologic processes, such as the atherosclerotic plaques, the amyloid plaques (the accumulation whereof among brain neurons is cause of the onset of the Alzheimer's disease) and the Lewy bodies (responsible for the Parkinson's disease).

Since the 60s, researchers such as Cartwright and Wintrobe have shown the high concentration of copper in various organs such as liver, brain, kidneys and pancreas.

Such capability of copper to represent a basic element in the development of various metabolic processes, led the first nuclear medicine researchers to introduce a radioactive isotope thereof (in particular, copper-64) in the study of metabolism: but such copper radionuclide (hereinafter identified with its chemical name “Cu”, whereas the relative radioactive isotope shall be identified as “64Cu”) had a development more on the aspect of scientific research than of medical application.

The Washington University of Saint Louise has introduced the 64Cu-ATSM in the study of the tumoural necrotic areas; more recently, scientific articles on the 64Cu-Dotatate for endocrine cancer and on the 64Cu-Trastuzumab for breast cancer have been published.

However, during these years, it has not been at all clarified the role of the 64Cu in its salt form Cl2 (64CuCl2, copper-64 chloride), aimed to a direct use of the cancer disease or of other pathologies.

It should be underlined that the term “direct use” means the use of the copper 64CuCl2 salt molecule alone, that is without the need of synthesizing a complex molecule, comprising the 64Cu bond to other chemical elements, such as in the case of the already known 64Cu-Dotatate and 64Cu-Trastuzumab.

The only studies on this matter, that is on the direct use of the 64Cu in its simple salt form, can be found in a pre-clinical work by Fangyu Peng, which demonstrates the possibility of its diagnostic (but non therapeutic) use in the prostate cancer.

The object of the present invention is to provide a pharmaceutical substance comprising 64Cu in its salt form which, per se and without bond with other complex molecules, may be employed in the treatment of certain pathologies, particularly of solid tumours, neuroendocrine tumours, skin tumours, leukaemias and lymphomas.

Another object of the present invention is to provide a pharmaceutical substance which, according to a first dosage, may be employed in the diagnosis of the Alzheimer's disease and, according to a second different dosage, finds use also in the therapeutic treatment of the same disease.

A further object of the present invention is to provide a procedure and suitable means for the production of such pharmaceutical substance.

These and other objects, which shall appear clearly hereinafter, are achieved with a drug according to claim 1, usable as therapeutic and diagnostic-therapeutic agent according to claims 5 to 21.

Other objects may also be achieved through the additional features of the dependent claims.

Further features and advantages of the present invention will appear more clearly from the following description of a preferred embodiment thereof, described hereinafter by way of a non-limiting example.

Hereinafter the present description, technical and scientific terms will be used, commonly known by the man skill in the art to which such description is addressed, and sufficiently clear for the invention to be implemented: however, when first mentioning a new element, component or unit of measure the complete name thereof shall be used, subsequently substituted by the relative abbreviation, acronym or brief chemical name when mentioned again.

According to the present invention, the chemical composition of the drug consists of copper-64 chloride (64CuCl2), that is copper-64 salt the chemical features thereof are clarified hereinafter.

1 mL of 64CuCl2 solution contains 925 MBq (Mega-Becquerels) of 64Cu at the calibration time of 01h00 a.m. CET (Central European Time) (where such calibration time is defined by the period between the end of the synthesis and the expiry time), corresponding to at least 0.25 μg (micrograms) of 64Cu. Every vial of the drug 64CuCl2 contains an activity range between 925 MBq and 2,775 MBq at the calibration time, corresponding to an accumulation between 0.25 and 0.75 μg, for a variable volume from 1 to 3 mL.

The minimum specific activity is 3.7 MBq of 64Cu for every μg of 64Cu, at the date and time of expiry.

The 64Cu, with an average life of 12.7 hours, has a Beta+emission of 17.6% with a maximum energy of 0.66 MeVs (Mega electron Volts), a Beta- emission of 38.5% with a maximum energy of 0.58 MeVs and an electron capture of 43.9%.

Such radionuclide 64Cu decays to stable nickel 64Ni at 61% with a Beta+ emission of 18% or with an electron capture of 43%. It also decays to stable zinc 64Zn with a Beta− emission at 39%.

The possible steps and the process means for obtaining the radionuclide 64Cu, preparatory to the subsequent achievement of the drug 64CuCl2, shall now be described in detail.

The production of 64CuCl2 takes place inside a stainless steel structure with an anti radiation shield: all the inner walls are in fact fully coated with stainless steel, with air tight execution, so as to allow an easy cleaning and decontamination of such structure.

On the front wall there is a suitable zone in plexiglass, which allows the vision of the inside of the structure and ensures the air tight.

Before obtaining the 64CuCl2, takes place the production of 64Cu using a cyclotron, an apparatus situated in a suitably shielded underground bunker and, through a dedicated transport line, in direct connection with the production laboratory.

The material subject to the action of the proton beam by said cyclotron is composed of stable nickel 64Ni, deposited on a gold disk.

Said bombardment action produces the 64Cu, according to the following nuclear reaction responsible for the transmutation: 64Ni (p, n) 64Cu.

The 64Cu thus obtained on the gold disk is transferred, even manually, to the above-mentioned structure, in a special container towards the shielded cell made of lead, where the synthesis module for the drug 64CuCl2 is located, and introduced into the first heating reactor.

On the contrary, the deposit of 64Ni, remaining on the gold disk, is dissolved with hydrochloric acid HCl 6N (6 mL), with subsequent formation of 64NiCl2 (nickel-64 chloride) and 64CuCl2.

The solution thus obtained is purified by making it pass through an ion exchange column, using the following fluxations:
two fluxations with 5 mL of HCl 6N, for removing nickel impurities,
one fluxation with 2 mL of HCl 0.5N, for reconditioning the ion exchange column and removing any cobalt present,
one fluxation with 3 mL of HCl 0.5N, for eluting copper.

The consequent 3 mL of the column (constituting the solution of 64CuCl2) are collected and evaporated to dryness; what remains therefrom is then reconstituted with 3 mL of HCl 0.1N.

The solution of 64CuCl2 thus obtained is finally transferred to the fractionator for the final dispensing thereof in vials, which represents the most suitable container for a drug of this type.

Such drug 64CuCl2 proves to be effective as a therapeutic agent for treating cancer, by triggering specific mechanisms against cancer cells.

Moreover, said drug 64CuCl2 proves to be effective both as a diagnostic agent and as a therapeutic agent against the cells affected by the Alzheimer's disease.

With regard to its action as an anti-cancer therapeutic agent, the uptake mechanism of the drug 64CuCl2 by the target cell is presumable to have the following process.

The 64CuCl2 enters the target cell passing the cellular membrane thereof (through methods still not perfectly known, but certainly implying the activation of the serotonin-threonine-kinase enzyme CTR1).

Inside said cell, the 64CuCl2 is used in an equilibrium of secretion and elimination processes of the copper-zinc superoxide dismutase (SOD) and cytochrome oxidase (COX) in the mitochondrions, partly binding to the DNA.

In fact, as known from the publication of Maskos and others (Acta Biochim Polonica 1981; 28:183-199), a bond of copper 2+ to the PH-dependent purinic bases takes place.

The 64CuCl2 plays a role of catalyst of the DNA replication reaction, and such role may also have a toxic effect when free radicals hydroxylated with hydrogen peroxide. H2O2 or with oxygen anion O- are generated.

Laboratory experimental tests have shown that such drug 64CuCl2, at a certain dosage, has an Auger radioactive effect that is capable of destroying the DNA of the cancer cell, in particular, reducing the volume of the tumoural lesions in solid tumours.

Said dosage is equal to a 1.5 Bq (Becquerel) for each cell, a quantity such that the therapeutic effectiveness of the drug is carried out through the expression of the toxicity thereof only inside the nucleus, achieving a true target therapy.

Such toxic effect of the drug 64CuCl2 towards the cancer cell is direct, through the above Auger effect, but also indirect, through the formation of free radicals which are introduced in the cytoplasm and amplify the toxic effect thereof on the DNA (this last feature is already known from studies of 2006 by Buchegger and others, however limited to works on copper intended as a chemical element and not as a radiopharmaceutical).

As said above, the unit dose per cell, required for the drug 64CuCl2 to carry out its anti-cancer therapeutic activity, is equal to 1.5 Bq/cell. The dose of the drug 64CuCl2 to be administered shall be suitably calculated based on such unit dose of 1.5 Bq/cell, according to the patient, the type of tumour and the extent of the latter.

By way of an example, it has been seen that the average dose for a standard pathology is about 3,700 MBq.

The administration protocol for the drug 64CuCl2 includes a first treatment with the therapeutic dose administered intravenously or arteriously loco-regionally (calculated according to the above-mentioned criteria), to be carried out on a first day and to be repeated on the following day.

Subsequently, the treatment shall be repeated every three months, according to the outcomes of the treatment. Of course the dosage intervals may be modulated and established according to the specific form of cancer treated and to the response of the patient to the drug.

The above described drug 64CuCl2 proved to be effective as a therapeutic agent for the following cancer pathologies: solid tumours (pancreas, prostate, brain, breast, ovary, head-neck, oesophagus, stomach, intestines, colon, liver, lung), neuroendocrine tumours, melanomas, leukaemias and lymphomas.

Laboratory experimental tests, carried out on about one hundred small-sized animals and verified via microPET, have confirmed a reduction in the tumoural mass between 30 and 60% compared to the starting tumoural mass, after only one treatment (according to the administration protocol described above).

Compared to other anti-cancer drugs, i.e. compared to chemotherapeutic agents or other radiotherapeutic agents, the drug 64CuCl2 object of this invention allows a number of advantages.

Compared to the drugs used in chemotherapy, said drug 64CuCl2 carries out a therapeutic treatment that is physical and not chemical, through the above Auger effect, by acting only within the nucleus of the target cell.

Moreover, being known the unit dose of drug per cell, it is possible to customise the therapeutic dosage for the specific case, through a dosimetric calculation.

Compared to other types of drugs used in radiotherapy, the drug 64CuCl2 does not need bonds with other complex molecules, as the other currently known radiopharmaceuticals; moreover; it acts directly on the DNA and the relative replication process.

The above is valid with reference to a use of the drug 64CuCl2 as a therapeutic agent in the treatment of cancer forms.

Nonetheless, as mentioned, said drug proved to be effective also against the Alzheimer's disease, both as a diagnostic agent and a therapeutic agent.

The two different uses are based on a different dosage of the drug, being it understood the identical unit dose per cell, equal to 1.5 Bq/cell.

More in particular, it has been seen that through the administration of an average dose of 370 MBq, the drug 64CuCl2 is capable of acting as a radiotracer for the diagnosis of the Alzheimer's disease.

In fact, it is capable of binding to the amyloid plaques, the accumulation whereof within the nerve cells characterises the presence of the pathology, and catalyses its formation process. By crossing the blood-brain barrier, the deposit of the subject drug is suitably identified inside said amyloid plaques, providing a diagnostic feedback beyond all doubts.

With the same uptake mechanism, but with an average dose administered substantially decupled (equal to 3,700 MBq), the drug 64CuCl2 carries out the function of therapeutic agent against the neuronal cells affected by the Alzheimer's disease.

In fact, the drug, by binding to said amyloid plaques, initially stabilises the growth thereof, to then slower it by braking such “drug-plaque” bonds.

The administration protocol for the drug 64CuCl2, as a therapeutic agent against the neuronal cells affected by the Alzheimer's disease, includes a first treatment with the therapeutic dose administered intravenously or arteriously loco-regionally (calculated according to the above-mentioned criteria), to be carried out on a first day and to be repeated on the following day.

Subsequently, the treatment shall be repeated every three months, according to the outcomes of the treatment. Of course the dosage intervals may be modulated and established according to the specific form of the Alzheimer's disease being treated and to the response of the patient to the drug.

It is clear that variants of the drug described above are possible to the man skilled in the art, without departing from the novelty scopes of the inventive idea, as well as it is clear that in the practical embodiment of the invention the various components described above may be replaced with technically equivalent ones.

1. Drug containing the salt form Cl₂ of copper-64 radioisotope ⁶⁴Cu, characterised in that
said drug copper-64 chloride ⁶⁴CuCl₂ is aimed to a direct use in patients, as a substance employable per se and without bond with other complex molecules,
and in that
each mL of solution of said drug ⁶⁴CuCl₂ contains 925 MBq of ⁶⁴Cu at the calibration time of 01h00 a.m. CET, corresponding to at least 0.25 micrograms of said ⁶⁴Cu.

2. Drug according to claim 1,
characterised in that
every vial of said drug ⁶⁴CuCl₂ has a volume between 1 and 3 mL and activity range between 925 and 2,775 MBq, corresponding to an accumulation of ⁶⁴Cu between at least 0.25 and 0.75 micrograms.

3. Drug according to claim 1,
characterised in that
its unit dose for effectiveness is equal to 1.5 Bq for every cell towards which said drug ⁶⁴CuCl₂ is targeted.

4-27. (canceled)

28. Drug according to claim 1,
characterised in that
it can be employed for the therapeutic treatment of a solid tumour, such as pancreatic, prostate, brain, breast, ovary, head-neck, oesophagus, stomach, intestines, colon, lung cancer.

29. Drug according to claim 28,
characterised in that it is administered intravenously or arteriously loco-regionally.

30. Drug according to claim 29,
characterised in that it is administered in a dosage customised for the specific patient wherein the dosage is on the average equal to 3,700 MBq.

31. Drug according to claim 28,
characterised in that it is administered a first time on a first day and a second time on the next day.

32. Drug according to claim 28,
characterised in that the administration is repeated every three months.

33. Drug according to claim 1,
characterised in that it can be employed for the treatment of a neuroendocrine tumour.

33. Drug according to claim 33,
characterised in that it is administered intravenously or arteriously loco-regionally.

35. Drug according to claim 34,
characterised in that it is administered in a dosage customised for the specific patient wherein the dosage is on the average equal to 3,700 MBq.

36. Drug according to claim 33,
characterised in that it is administered a first time on a first day and a second time on the next day.

37. Drug according to claim 33,
characterised in that the administration is repeated every three months.

38. Drug according to claim 1,
characterised in that it can be employed for the treatment of a melanoma.

39. Drug according to claim 38,
characterised in that it is administered intravenously or arteriously loco-regionally.

40. Drug according to claim 39,
characterised in that it is administered in a dosage customised for the specific patient wherein the dosage is on the average equal to 3,700 MBq.

41. Drug according to claim 38,
characterised in that it is administered a first time on a first day and a second time on the next day.

42. Drug according to claim 38,
characterised in that the administration is repeated every three months.

43. Drug according to claim 1,
characterised in that
it can be employed for the treatment of leukaemia or a lymphoma.

44. Drug according to claim 43,
characterised in that
it is administered intravenously or arteriously loco-regionally.

45. Drug according to claim 44,
characterised in that it is administered in a dosage customised for the specific patient wherein the dosage is on the average equal to 3,700 MBq.

46. Drug according to claim 43,
characterised in that it is administered a first time on a first day and a second time on the next day.

47. Drug according to claim 43, characterised in that the administration is repeated every three months.

48. Drug according to claim 1,
characterised in that
it can be employed for the diagnosis of the Alzheimer's disease.

49. Drug according to claim 48,
characterised in that
it is administered intravenously or arteriously loco-regionally.

50. Drug according to claim 49,
characterised in that it is administered in a dosage customised for the specific patient wherein the dosage is on the average equal to 370 MBq.

51. Drug according to claim 48,
characterised in that it is administered a first time on a first day and a second time on the next day.

52. Drug according to claim 48,
characterised in that the administration is repeated every three months.

53. Drug according to claim 1,
characterised in that
it can be employed for the therapeutic treatment of the Alzheimer's disease.

54. Drug according to claim 53,
characterised in that
it is administered intravenously or arteriously loco-regionally.

55. Drug according to claim 54,
characterised in that it is administered in a dosage customised for the specific patient wherein the dosage is on the average equal to 3,700 MBq.

56. Drug according to claim 53,
characterised in that it is administered a first time on a first day and a second time on the next day.

57. Drug according to claim 53,
characterised in that the administration is repeated every three months.
